(19) United States
(12) Patent Application Publication
(10) Pub. No.: US 2007/0197599 A1
(43) Pub. Date: Aug. 23, 2007

(54) HYDROXYLAMINES AND DERIVATIVES AS ANTI-ANGIOGENIC AGENTS

(76) Inventors: William L. Matier, Hockessin, DE (US); Ghanshyam Patil, Lincoln University, PA (US)

Correspondence Address: WOODCOCK WASHBURN LLP, CIRA CENTRE, 12TH FLOOR, 2929 ARCH STREET, PHILADELPHIA, PA, 19104-2891, US

(21) Appl. No.: 11/669,885

(22) Filed: Jan. 31, 2007

Related U.S. Application Data
(60) Provisional application No. 60/764,432, filed on Feb. 2, 2006.

(51) Int. Cl. A61K 31/445; A61K 31/421

(52) U.S. Cl. 514/327; 514/376

(57) ABSTRACT

The present disclosure provides compounds that include hydroxylamines and ester derivatives thereof and methods for using the same for the treatment of angiogenesis and related diseases.

### CROSS REFERENCE TO RELATED APPLICATION

This application claims the benefit of U.S. Provisional Application Ser. No. 60/764,432, filed Feb. 2, 2006, the disclosure of which is incorporated herein by reference in its entirety.

### BACKGROUND OF THE INVENTION

Various patents and other publications are referenced herein. The contents of each of these patents and publications are incorporated by reference herein, in their entireties. The entire contents of commonly-owned co-pending U.S. Publication Nos. 2004/0002461, 2005/0130906 and 2005/0131025 are incorporated by reference herein.

Angiogenesis is a complex process of new blood vessel development and formation. Angiogenesis occurs in response to specific signals and involves a complex process characterized by infiltration of the basal lamina by vascular endothelial cells in response to angiogenic growth signal(s), degradation of extracellular matrix and migration of the endothelial cells toward the source of the signal(s), and subsequent proliferation and formation of the capillary tube. Blood flow through the newly formed capillary is initiated after the endothelial cells come into contact and connect with a preexisting capillary.

Angiogenesis is highly regulated and involves a balancing between various angiogenic stimulators and inhibitors. Normally, for mature individuals, there is not much new vessel formation, which means that the naturally occurring balance between endogenous stimulators and inhibitors of angiogenesis heavily favors the inhibitors. Rastinejad et al., 1989, Cell 56:345-355. However, there are some instances in which neovascularization occurs under normal physiological conditions, such as wound healing, organ regeneration, embryonic development, and female reproductive processes, but the angiogenesis is stringently regulated and spatially and temporally delimited. On the other hand, under conditions of pathological angiogenesis, such as that characterizing solid tumor growth, these regulatory controls fail.

When the regulatory controls are compromised and unregulated angiogenesis becomes pathologic, this can lead to sustained progression of many neoplastic and non-neoplastic diseases. A number of serious diseases are dominated by abnormal neovascularization and include solid tumor growth and metastases, arthritis, some types of eye disorders, and psoriasis. See, e.g., reviews by Moses et al., 1991, Biotech. 9:630-634; Folkman et al., 1995, N. Engl. J. Med., 333:1757-1763; Auerbach et al., 1985, J. Microvasc. Res. 29:401-411; Folkman, 1985, Advances in Cancer Research, eds. Klein and Weinhouse, Academic Press, New York, pp. 175-203; Patz, 1982, Am. J. Opthalmol. 94:715-743; and Folkman et al., 1983, Science 221:719-725. As with healthy tissue, tumors require blood vessels to sustain the underlying cells. In a number of pathological conditions, the process of angiogenesis can even contribute to the disease state. Indeed, some investigators have suggested that the growth of solid tumors is dependent on angiogenesis. Folkman and Klagsbrun, 1987, Science 235:442-447.

Reactive oxygen species (ROS), such as superoxide and hydrogen peroxide, have been reported to induce angiogenesis in vivo, possibly through up-regulation of inducible nitric oxide synthase and increased production of endogenous nitric oxide. Polytarchou & Papadimitriou, 2005, Eur. J. Pharmacol. 510:31-38. ROS have also been reported to stimulate vascular endothelial growth factor (VEGF) release, and mediate activation of a MAP kinase (Mitogen Activated Protein Kinases) signaling pathway for VEGF. Kuroki et al., 1996, J. Clin. Invest. 98:1667-1675; Cho et al., 2001, Am. J. Physiol. Heart Circ. Physiol. 280: H2357-H2363.

Certain antioxidants have also been shown to have angiogenesis inhibiting activity, for example, superoxide dismutase and the nitroxide TEMPOL, but not the reduced product of TEMPOL, the hydroxylamine TEMPOL-H. Other anti-angiogenic agents include VEGF antagonists, bFGF antagonists, or nitric oxide synthase (NOS) antagonists, such as N^{ω}-nitro-L-arginine methyl ester (L-NAME) and dexamethasone.

Nitroxides such as TEMPOL have been of greater interest because of their radical scavenging properties and exertion of an anti-inflammatory effect in various animal models of oxidative damage and inflammation. Nilsson et al. disclosed, in WO 88/05044, that nitroxides and their corresponding hydroxylamines are useful in prophylaxis and treatment of ischemic cell damage, presumably due to antioxidant effects. Paolini et al. (U.S. Pat. No. 5,981,548) disclosed N-hydroxylpiperidine compounds and their potential general utility in the treatment of pathologies arising from oxygen radicals and as foodstuff and cosmetic additives. Hsia et al. (U.S. Pat. Nos. 6,458,758, 5,840,701, 5,824,781, 5,817,632, 5,807,831, 5,804,561, 5,767,089, 5,741,893, 5,725,839 and 5,591,710) disclosed the use of stable nitroxides and hydroxylamines (e.g., TEMPOL and its hydroxylamine counterpart, TEMPOL-H), in combination with a variety of biocompatible macromolecules, to alleviate free radical toxicity in blood and blood components. Hahn et al. (1998, Int. J. Radiat. Oncol. Biol. Physics 42: 839-842; 2000, Free Rad. Biol. Med. 28: 953-958) reported on the in vivo radioprotection and effects on blood pressure of the stable free radical nitroxides and certain hydroxylamine counterparts.

Due to their comparative lack of toxicity, hydroxylamines are preferable to nitroxides as therapeutic agents. Published United States Patent Applications 2004/0002461, 2005/0130906 and 2005/0131025 to Matier and Patil disclose hydroxylamines and related compounds and their use in the treatment of a variety of ophthalmic conditions in which oxidative damage or inflammation are involved. Such compounds possess numerous advantageous qualities, including robust anti-inflammatory and antioxidant activities, as well as ocular permeability in some instances. However, while some nitroxides, e.g., TEMPOL, have demonstrated some anti-angiogenic activity, hydroxylamines heretofore have not been reported as possessing any anti-angiogenic activity.

### SUMMARY OF THE INVENTION

The current disclosure details methods of inhibiting pathological angiogenesis in a patient by administering to the patient a hydroxylamine compound or an ester derivative thereof in a therapeutically sufficient amount to inhibit pathological angiogenesis. The ester derivatives of the hydroxylamines have the formula I:

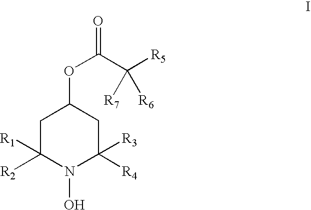

wherein R₁ and R₂ are, independently, H or C₁ to C₃ alkyl; R₃ and R₄ are, independently C₁ to C₃ alkyl; and wherein R₁ and R₂, taken together, or R₃ and R₄, taken together, or both are cycloalkyl; R₅ is H, OH, or C₁ to C₆ alkyl; R₆ is or C₁ to C₆ alkyl, alkenyl, alkynyl, or substituted alkyl or alkenyl; R₇ is C₁ to C₆ alkyl, alkenyl, alkynyl, or substituted alkyl or alkenyl; wherein R₆ and R₇, or R₅, R₆ and R₇, taken together, form a carbocycle or heterocycle having from 3 to 7 atoms in the ring.

Further, the disclosure provides methods of treating a patient having a disease state that involves pathological angiogenesis by administering to the patient the hydroxylamine compound or an ester derivative thereof in a therapeutically sufficient amount to inhibit pathological angiogenesis. The ester derivatives of the hydroxylamines have the formula I. In some embodiments, these methods further include co-administering an additional agent, such as an antioxidant, a reducing agent, an additional anti-angiogenic agent, or an antineoplastic agent.

According to other aspects of the invention, pharmaceutical compositions comprising the aforementioned hydroxylamines or ester derivatives are provided for the treatment of disease states in which angiogenesis is involved.

Other features and advantages of the invention will be understood by reference to the drawings, detailed description and examples that follow.

### BRIEF DESCRIPTION OF THE DRAWINGS

FIG. 1. Disappearance of Compound 1 (Cyclopropanecarboxylic acid 1-hydroxy-2,2,6,6-tetramethyl-piperidin-4-yl ester) in rat, rabbit, dog, and human plasma as a function of incubation time under standardized incubation conditions.

FIG. 2. Appearance of TPH in rat, rabbit, dog, and human plasma as a function of Compound 1 Incubation Time Under Standardized Incubation Conditions.

FIG. 3. Disappearance of Compound 1 and appearance of TPH in rat plasma as a function of incubation time.

FIG. 4. Disappearance of Compound 1 and appearance of TPH in rabbit plasma as a function of incubation time.

FIG. 5. Disappearance of Compound 1 and appearance of TPH in dog plasma as a function of incubation time.

FIG. 6. Disappearance of Compound 1 and appearance of TPH in human plasma as a function of incubation time.

FIG. 7. Mean±SD plasma concentrations of Compound 1 as a function of time in Sprague-Dawley Rats (n=5-6) after a single 1 0-minute intravenous infusion of Compound 1.

FIG. 8. Mean±SD plasma concentrations of TPH as a function of time in Sprague-Dawley Rats (n=5-6) after a single 10-minute intravenous infusion of Compound 1.

### DETAILED DESCRIPTION OF ILLUSTRATIVE EMBODIMENTS

The present invention provides methods for the treatment or prevention of a number of diseases and disorders in which pathogenic angiogenesis is an underlying causal factor. The methods comprise administration of compositions comprising a pharmaceutically acceptable carrier or diluent and a hydroxylamine compound, or ester derivative thereof, in a therapeutically sufficient amount to prevent, retard the development of or reduce the symptoms of one or more angiogenesis-associated diseases or conditions.

As used herein, the term “angiogenesis” means the generation of new blood vessels into a tissue or organ. Under normal physiological conditions, humans or animals undergo angiogenesis only in very specific restricted situations. For example, angiogenesis is normally observed in wound healing, fetal and embryonal development and formation of the corpus luteum, endometrium and placenta. The term “endothelium” is defined herein as a thin layer of flat cells that lines serous cavities, lymph vessels, and blood vessels. These cells are defined herein as “endothelial cells”. The term “endothelial inhibiting activity” means the capability of a molecule to inhibit angiogenesis in general. The inhibition of endothelial cell proliferation at various stages also results in an inhibition of angiogenesis (Albo, et al., 2004, Curr Pharm Des. 10(1):27-37).

Many diseases or adverse conditions are associated with angiogenesis. Examples of such diseases or disorders include, but are not limited to, (1) neoplastic diseases, such as cancers of the breast, head, rectum, gastrointestinal tract, lung, bronchii, pancreas, thyroid, testicles or ovaries, leukemia (e.g., acute myelogenous leukemia), sinonasal natural killer/T-cell lymphoma, malignant melanoma, adenoid cystic carcinoma, angiosarcoma, anaplastic large cell lymphoma, endometrial carcinoma,or prostate carcinoma (2) hyperproliferative disorders, e.g., disorders caused by non-cancerous (i.e. non-neoplastic) cells that overproduce in response to a particular growth factor, such as psoriasis, endometriosis, atherosclerosis, systemic lupus and benign growth disorders such as prostate enlargement and lipomas; (3) cell proliferation as a result of infectious diseases, such as Herpes simplex infections, Herpes zoster infections, protozoan infections and Bartonellosis (a bacterial infection found in South America); (4) arthritis, including rheumatoid arthritis and osteoarthritis; (5) chronic inflammatory disease, including ulcerative colitis and Crohn's disease; and (6) other conditions, including the childhood disease, hemangioma, as well as hereditary diseases such as Osler-Weber-Rendu disease, or hereditary hemorrhagic telangiectasia.

The present inventors have determined that angiogenesis, and the diseases or disorders involving angiogenesis, can be ameliorated through the administration of hydroxylamine compounds such as TEMPOL-H (TPH, as well as ester derivatives of such compounds that may be hydrolyzable to form hydroxylamine compounds. This determination was made in part through the use of the chick chorioallantoic membrane (CAM) model of angiogenesis, the protocols of which are set forth in the examples.

While it has been shown in some instances that the nitroxide TEMPOL inhibits hydrogen peroxide-induced angiogenesis, anti-angiogenic activity of hydroxylamines has not been demonstrated prior to the present invention. In addition, heretofore there has been no suggestion that nitroxides or hydroxylamines could prevent VEGF or bFGF growth factor-induced angiogenesis. Nor would such activity of hydroxylamines be predicted, inasmuch as nitroxides such as TEMPOL, and their hydroxylamine counterparts such as TEMPOL-H, possess very different molecular structural appearances, physical constants and chemical characteristics. For example, it has been reported that TEMPOL-mediated radioprotection of mouse V79 cells was concentration dependent, but the hydroxylamine, TEMPOL-H, did not provide any radioprotection (Mitchell et al., 2000, Radiation, Radicals, and Images; Annals of the New York Academy of Sciences 899:28-43). Additionally, TEMPOL, but not TEMPOL-H, prevented X-ray radiation damage to lens endothelial cells in vitro (Sasaki, et al., 1998, Invest Ophthalmol Vis Sci. 39(3):544-52.). Similarly, it has been found that TEMPOL was not effective in preventing selenite induced cataract in mice, but TEMPOL-H was effective in that model. Further, nitroxides such as TEMPOL have been found to be cytotoxic, and sometimes act as a prooxidant instead of an antioxidant (Glebska et al., 2003, Free Radical Biol. Med. 35: 310-316). For these and other reasons, the anti-angiogenic effect of TEMPOL against H₂O₂-induced angiogenesis is not predictive that hydroxylamines would possess such activity. In addition, as mentioned above, there is no precedent for the prevention of growth factor-induced angiogenesis by either TEMPOL or hydroxylamines.

Preferred examples of the type of hydroxylamine compounds suitable for use in the present invention are TEMPOL-H (TPH, the hydroxylamine reduced form of the nitroxide 4-hydroxy-2,2,6,6-tetramethylpiperidin-1-yloxy), TEMPO-H (the hydroxylamine reduced form of the nitroxide 2,2,6,6-tetramethylpiperidin-1-yloxy) and OXANO-H (2-Ethyl-2,4,4-trimethyl-oxazolidin-3-ol), which is the reduced form of OXANO, 2-ethyl-2,4,4-trimethyloxazolidin-3-yloxy). Other hydroxylamine compounds suitable for use in the present invention include, but are not limited to, those disclosed by Hahn et al. (1998, supra; 2000, supra), Samuni et al. (2001, supra); and in U.S. Pat. No. 5,981,548 to Paolini, et al. (disclosing certain N-hydroxylpiperidine esters and their use as antioxidants in a number of contexts); U.S. Pat. No. 4,404,302 to Gupta et al. (disclosing the use of certain N-hydroxylamines as light stabilizers in plastics formulations); U.S. Pat. No. 4,691,015, to Behrens et al. (describing hydroxylamines derived from hindered amines and the use of certain of them for the stabilization of polyolefins); the hydroxylamine compounds disclosed in the several aforementioned U.S. patents to Hsia et al.; and the hydroxylamine counterparts of the nitroxides disclosed in U.S. Pat. Nos. 5,462,946 and 6,605,619 to Mitchell et al., namely, (1) compounds of the formula R₃—N(R₄)(R₅) wherein R₃ is —OH and R₄ and R₅ combine together with the nitrogen to form a heterocycle group, or wherein R₄ and R₅ themselves comprise a substituted or unsubstituted cyclic or heterocyclic group; (2) metal-independent hydroxylamines of formula R₃—N(R₄)(R₅) wherein R₃ is —OH and R₄ and R₅, together with the nitrogen atom to which they are bonded, form a 5- or 6-membered heterocyclic group, which, in addition to said nitrogen atom, comprises one or more heteroatoms selected from the group consisting of oxygen, nitrogen and sulfur, or R₄ and R₅, separately, each comprise a substituted or unsubstituted 5- or 6-membered cyclic group or a substituted or unsubstituted 5- or 6-membered heterocyclic group, which comprises one or more heteroatoms selected from the group consisting of oxygen, nitrogen and sulfur; or (3) oxazolidine compounds of the formula:

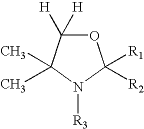

wherein R₁ is —CH₃ and R₂ is —C₂H₅, —C₃H₇, —C₄H₉, —C₅H₁₁, —C₆H₁₃, —CH₂CH(CH₃)₂, —CHCH₃C₂H₅, or —(CH₂)₇CH₃, and R₃ is —OH, or wherein R₁ and R₂ together form spirocyclopentane, spirocyclohexane, spirocycloheptane, spirocyclooctane, 5-cholestane or norbornane; and pharmaceutically acceptable salts of any of the above-listed compounds. Insofar as is known the above-referenced compounds have not been used heretofore for inhibiting angiogenesis.

Ester derivatives of hydroxylamines suitable for use in the present invention comprise compounds of formula I or their pharmaceutically acceptable salts, examples of which are described in detail in U.S. Published Application 2004/0002461:

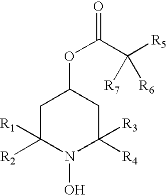

where R₁ and R₂ are, independently, H or C₁ to C₃ alkyl;

R₃ and R₄ are, independently C₁ to C₃ alkyl; or

where R₁ and R₂, taken together, or R₃ and R₄, taken together, or both may be cycloalkyl;

R₅ is H, OH, or C₁ to C₆ alkyl;

R₆ is C₁ to C₆ alkyl, alkenyl, alkynyl, or substituted alkyl or alkenyl;

R₇ is C₁ to C₆ alkyl, alkenyl, alkynyl, substituted alkyl, alkenyl, cycloalkyl, or heterocycle;

or where R₆ and R₇, or R₅, R₆ and R₇, taken together, form a carbocycle or heterocycle having from 3 to 7 atoms in the ring.

The methods of the present invention may also utilize compositions comprising a pharmaceutically acceptable carrier or diluent and a hydroxylamine compound having an N-hydroxy piperidine portion bound to a solubility modifying portion, the compound having a solubility in water at 25° C. of at least about 0.25% by weight and a water/n-octanol partition coefficient at 25° C. of at least about 5. The composition may have the N-hydroxy piperidine portion cleavable from the compound under conditions found in biological tissues, such as found in the eye. The N-hydroxy piperidine portion may be cleaved enzymatically. The compositions may also exist wherein the N-hydroxy piperidine portion is 1-oxyl-4-hydroxy-2,2,6,6-tetramethylpiperidyl.

The term C₁ to Cₙ alkyl, alkenyl, or alkynyl, in the sense of this invention, means a hydrocarbyl group having from 1 to n carbon atoms in it, wherein n is an integer from 1 to about 20, preferably 1 to about 10, yet more preferably, 1 to about 6, with from 1 to about 3 being even more preferred. The term thus comprehends methyl, ethyl, n-propyl, iso-propyl, n-butyl, sec-butyl, iso-butyl, tert-butyl, and the various isomeric forms of pentyl, hexyl, and the like. Likewise, the term includes ethenyl, ethynyl, propenyl, propynyl, and similar branched and unbranched unsaturated hydrocarbon groups of up to n carbon atoms. As the context may admit, such groups may be functionalized such as with one or more hydroxy, alkoxy, alkylthio, alkylamino, dialkylamino, aryloxy, arylamino, benzyloxy, benzylamino, heterocycle, or YCO-Z, where Y is O, N, or S and Z is alkyl, cycloalkyl, heterocycle, or aryl substituent.

The term carbocycle defines cyclic structures or rings, wherein all atoms forming the ring are carbon. Exemplary of these are cyclopropyl, cyclobutyl, cyclopentyl, cyclohexyl, cycloheptyl, etc. Cyclopropyl is one preferred species. Heterocycle defines a cyclic structure where at least one atom of the ring is not carbon. Examples of this broad class include furan, dihydrofuran, tetrahydrofuran, pyran, oxazole, oxazoline, oxazolidine, imidazole and others, especially those with an oxygen atom in the ring. Five, six and seven membered rings with at least one oxygen or nitrogen atom in the ring are preferred heterocycles. Furanyl and tetrahydrofuranyl species are among those preferred.

It is preferred for certain embodiments that each of R₁ through R₄ be lower alkyl that is C₁ to C₃ alkyl. Preferably, all these groups are methyl for convenience in synthesis and due to the known efficacy of moieties having such substitution at these positions. However, other substituents may be used as well.

In certain embodiments, compounds are employed where R₆ is C₁ to C₆ alkyl substituted with at least one C₁ to C₆ alkoxy or benzyloxy group. Preferred among these are compounds having ethoxy or benzyloxy substituents. Among preferred compounds are those where each of R₁ through R₄ is methyl, R₅ is H or methyl, R₆ is methyl substituted with benzyloxy or C₁ to C₆ alkoxy, and R₇ is methyl or where R₆ and R₇ form a cyclopropyl group as well as the compound in which each of R₁ through R₄ is methyl, R₅ is methyl, R₆ is ethoxy or benzyloxy methyl, and R₇ is methyl. An additional preferred compound is one in which each of R₁ through R₄ is methyl, R₅ is methyl, R₆ is hydroxymethyl, and R₇ is methyl.

Other useful compounds are those wherein each of R₁ through R₄ is methyl, and R₅, R₆, and R₇ form a furanyl group, or in which R₆ and R₇ form a tetrahydrofuranyl group. The compound where R₁ through R₄ is methyl, R₅ is H and, R₆ and R₇ form a cyclopropyl ring is a further preferred. Examples of compounds useful in the methods of the present invention include, but are not limited to those described in U.S. Patent Publication No. US 2004/0002461A1, such as 1-oxyl-4-(3′-ethoxy-2′,2′-dimethyl)propanecarbonyloxy-2,2,6,6-tetramethylpiperidine; 1-hydroxy-4-(3′-ethoxy-2′,2′-dimethyl)propanecarbonyloxy-2,2,6,6-tetramethylpiperidine hydrochloride; 1 -oxyl-4-cyclopropanecarbonyloxy-2,2,6,6-tetramethylpiperidine; 1-hydroxy-4-cyclopropanecarbonyloxy-2,2,6,6-tetramethylpiperidine hydrochloride; 1-oxyl-4-(3′-benzyloxy-2′,2′-dimethyl)propanecarbonyloxy-2,2,6,6-tetramethylpiperidine; 1-hydroxy-4-(3′-benzyloxy-2′,2′-dimethyl)propanecarbonyloxy-2,2,6,6-tetramethylpiperidine hydrochloride; 1-hydroxy-4-(3′-hydroxy-2′,2′-dimethyl)propanecarbonyloxy-2,2,6,6-tetramethylpiperidine hydrochloride; 1-oxyl-4-(1-methyl-cyclopropane)carbonyloxy-2,2,6,6-tetramethylpiperidine; 1-hydroxy-4-(1-methyl-cyclopropane)carbonyloxy-2,2,6,6-tetramethylpiperidine hydrochloride; 1-oxyl-4-(2-furan)carbonyloxy-2,2,6,6-tetramethylpiperidine; 1-hydroxy-4-(2′-furan)carbonyloxy-2,2,6,6-tetramethylpiperidine hydrochloride; 1-oxyl-4-(3′-tetrahydrofuran)carbonyloxy-2,2,6,6-tetramethylpiperidine; 1-hydroxy-4-(3′-tetrahydrofuran)carbonyloxy-2,2,6,6-tetramethylpiperidine hydrochloride. 1-hydroxy-4-cyclopropanecarbonyloxy-2,2,6,6-tetramethylpiperidine hydrochloride, referred to herein as Compound 1, is particularly preferred.

While not wishing to be bound by theory, Applicants believe that Compound 1 (compound of formula 1, wherein R¹, R², R³, and R⁴ are methyl, R⁵ is H, and R⁶ and R⁷ taken together form a cyclopropane ring) and the other compounds of formula I are believed exert their anti-angiogenic and other therapeutic effects in two ways. First, the ester compounds are hydrolyzed in situ to form hydroxylamine components that exert therapeutic activity. Second, the esterified compounds themselves possess antioxidant activity, and therefore may possess anti-angiogenic activity, thereby supporting the therapeutic efficacy of pharmaceutical preparations comprising the compounds.

In connection with the first basis for activity of the compounds of formula I, i.e., cleavage to liberate hydroxylamine components, numerous esterases are known to be present in various tissues and organs of the body, and particularly in ocular tissues, especially the cornea. The specific esterase(s) that cleaves the esters of the present series need not be identified in order to practice the invention. The cleavage of the esters occurs rapidly and essentially completely on administering the compounds to the eyes of rabbits. This is shown by the presence of TEMPOL-H in the aqueous humor at all times (30, 60, 90 and 120 minutes) examined after topical dosing. In contrast, the esters are stable in aqueous solutions in the absence of such esterases. The cleavage of the esters has also been demonstrated in plasma of various animal species. As described in Example 5, the in-vitro half-life of an ester derivative of TEMPOL-H (TPH) in rat, rabbit, dog, and human plasma was measured. The disappearance of the derivative was quantitatively accounted for, on a molar basis, by the formation of TEMPOL-H.

Compositions in accordance with the methods of the invention are formulated and administered so as to apply a dosage effective for exerting an anti-angiogenic effect in a target tissue. The amount of hydroxylamine or derivative can range from about 0.1% to about 25% weight by volume in the formulation, or a corresponding amount by weight. In some embodiments, it is preferable that the active drug concentration be 0.25% to about 25%. The concentration of the hydroxylamine component will preferably be in the range of about 0.1 μM to about 10 mM in the tissues and fluids. In some embodiments, the range is from 1 μm to 5 mM, in other embodiments the range is about 10 μM to 2.5 mM. In other embodiments, the range is about 50 μM to 1 mM. Most preferably the range of hydroxylamine concentration will be from 1 to 100 μM. In embodiments that include a reducing agent, either within the formulation or administered separately. The concentration of the reducing agent will be from 1 μM to 5 mM in the tissues and fluids, preferably in the range of 10 μM to 2 mM. The concentrations of the components of the composition are adjusted appropriately to the route of administration, by typical pharmacokinetic and dilution calculations, to achieve such local concentrations.

The compositions utilized in accordance with the inventive methods may contain more than one hydroxylamine compound. In some embodiments, two or more hydroxylamines are administered simultaneously. In other embodiments, they are administered sequentially.

Further, the methods of the invention include combination therapy. In some embodiments of the invention, the hydroxylamines or derivatives are administered with another compound known in the art that is useful for treating a disease or disorder associated with pathogenic angiogenesis. The other compound(s) known in the art may be administered simultaneously with the hydroxylamine compounds, or may be administered sequentially.

For example, the hydroxylamine compounds can be administered in combination with one or more additional anti-angiogenic agents. In general, anti-angiogenic agents can be any known inhibitor or down regulator of an angiogenic agent or an inhibitor of the cell signaling pathway promoted by an angiogenic agent, including, but not limited to, cartilage-derived factors, angiostatic steroids, angiostatic vitamin D analogs, angiostatin, endostatin, and verostatin. There are some anti-angiogenic agents that are thought to affect a specific angiogenic factor, e.g., the angiogenic factor angiogenin. Anti-angiogenic agents specific for angiogenin include monoclonal antibodies that bind angiogenin, human placental ribonuclease inhibitor, actin, and synthetic peptides corresponding to the C-terminal region of angiogenin. Anti-angiogenic agents of microbial origin are also contemplated herein. Such agents include anthracycline, 15-deoxyspergualin, D-penicillamine, eponemycin, fumagillin, herbimycin A, rapamycin and neomycin. The term “neomycin” refers to an antibiotic complex composed of neomycins A, B and C, which together is also known as Mycifradin, Myacyne, Fradiomycin, Neomin, Neolate, Neomas, Nivemycin, Pimavecort, Vonamycin Powder V, and analogs thereof.

The compositions may further include one or more antioxidants. Exemplary reducing agents include mercaptopropionyl glycine, N-acetylcysteine, β-mercaptoethylamine, glutathione, ascorbic acid and its salts, sulfite, or sodium metabisulfite, or similar species. In addition. antioxidants can also include natural antioxidants such as vitamin E, C, leutein, xanthine, beta carotene and minerals such as zinc and selenium.

The pharmaceutical compositions of the invention may optionally comprise one or more anti-neoplastic agents, which include, but are not limited to, alkaloids such as docetaxel, etoposide, trontecan, paclitaxel, teniposide, topotecan, vinblastine, vincristine, and vindesine; alkylating agents such as busulfan, improsulfan, piposulfan, aziridines, benzodepa, carboquone, meturedepa, uredepa, altretamine, triethylenemelamine, triethylenephosphoramide, triethylenethiophosphoramide, chlorambucil, chloraphazine, cyclophosphamide, estramustine, ifosfamide, mechlorethamine, mechlorethamine oxide hydrochloride, melphalan, novembichin, perfosfamide, phenesterine, prednimustine, trofosfamide, uracil mustard, carmustine, chlorozotocin, fotemustine, lomustine, nimustine, ranimustine, dacarbazine, mannomustine, mitobronitol, mitolactol, pipobroman, temozolomide; antibiotics and analogues such as aclacinomycinsa actinomycin F₁, anthramycin, azaserine, bleomycins, cactinomycin, carubicin, carzinophilin, chromomycins, dactinomycin, daunorubicin, 6-diazo-5-oxo-L-norleucine, doxorubicin, epirubicin, idarubicin, menogaril, mitomycins, mycophenolic acid, nogalamycin, olivomycins, peplomycin, pirarubicin, plicamycin, porfiromycin, puromycin, streptonigrin, streptozocin, tubercidin, zinostatin, zorubicin; antimetabolites such as denopterin, edatrexate, methotrexate, piritrexim, pteropterin, Tomudex®, trimetrexate, cladribine, fludarabine, 6-mercaptopurine, thiamiprine, thioguanine, ancitabine, azacitidine, 6-azauridine, camofur, cytarabine, doxifluridine, emitefur, enocitabune, floxuridine, fluorouracil, gemcitabine, tegafur; L-Asparaginase; immunomodulators such as interferon-.alpha., interferon-.beta., interferon-.gamma., interleukin-2, lentinan, propagermanium, PSK, roquinimex, sizofican, ubenimex; platimum complexes such as carboplatin, cisplatin, miboplatin, oxaliplatin; aceglarone; amsacrine; bisantrene; defosfamide; demecolcine; diaziquone; eflornithine; elliptinium acetate; etoglucid; fenretinide; gallium nitrate; hydroxyurea; lonidamine; miltefosine; mitoguazone; mitoxantrone; mopidamol; nitracine; pentostain; phenamet; podophyllinic acid 2-ethyl-hydrazide; procabazine; razoxane; sobuzoxane; spirogermanium; tenuzonic acid; triaziquone; 2,2′,2″trichlorotriethylamine; urethan; antineoplastic hormone or analogues such as calusterone, dromostanolone, epitiostanol, mepitiostane, testolacone, aminoglutethimide, mitotane, trilostane, bicalutamide, flutamide, nilutamide, droloxifene, tamoxifen, toremifene, aminoglutethimide, anastrozole, fadrozole, formestane, letrozole, fosfestrol, hexestrol, polyestradiol phosphate, buserelin, goserelin, leuprolide, triptorelin, chlormadinone acetate, medroxyprogesterone, megestrol acetate, melengestrol; porfimer sodium; batimastar; and folinic acid. For a description of these and other antineoplastic agents that may comprise the pharmaceutical composition of the invention, see The Merck Index, 12th ed.

Pathological angiogenesis or proliferation of endothelial cells has been associated with many diseases or conditions, including hyperproliferative and neoplastic diseases and inflammatory diseases and disorders, as listed in detail above. The methods of the invention may be adapted for the treatment of any condition in which angiogenesis is a causal factor. Compositions can be administered by any of the routes conventionally used for drug administration. Such routes include, but are not limited to, oral, topical parenteral and by inhalation. Parenteral delivery may be intraperitoneal, intravenous, perioral, subcutaneous, intramuscular, intraarterial, etc. The disclosed compositions can be administered in conventional dosage forms prepared by combining with standard pharmaceutically acceptable carriers according to procedures known in the art. Such combinations may involve procedures such as mixing, granulating, compressing and dissolving the appropriate ingredients.

The form and nature of the pharmaceutically acceptable carrier is controlled by the amounts of the active ingredient to which it is combined, the route of the administration, and other well-known variables. The active ingredient can be one of the present compounds, i.e., hydroxylamines or the ester derivatives thereof. As used herein, the term “carrier” refers to diluents, excipients and the like for use in preparing admixtures of a pharmaceutical composition. The term “pharmaceutically acceptable” means approved by a regulatory agency of the Federal or a state government or listed in the U.S. Pharmacopeia or other generally recognized pharmacopeia for use in animals, and more particularly in humans. Such pharmaceutically acceptable carriers or diluents and methods for preparing are well known in the art (see, e.g., Remington's Pharmaceutical Sciences, Meade Publishing Col., Easton, Pa., latest edition; the Handbook of Pharmaceutical Excipients, APhA publications, 1986).

Pharmaceutically acceptable carriers may be, for example, a liquid or solid. Liquid carriers include, but are not limited, to water, saline, buffered saline, dextrose solution, preferably such physiologically compatible buffers as Hank's or Ringer's solution, physiological saline, a mixture consisting of saline and glucose, and heparinized sodium-citrate-citric acid-dextrose solution and the like, preferably in sterile form. Exemplary solid carrier include agar, acacia, gelatin, lactose, magnesium stearate, pectin, talc and like.

In some of the embodiments, the compositions can be administered orally. For such administrations, the pharmaceutical composition may be in liquid form, for example, solutions, syrups or suspensions, or may be presented as a drug product for reconstitution with water or other suitable vehicle before use. Such liquid preparations may be prepared by conventional means with pharmaceutically acceptable additives such as suspending agents (e.g., sorbitol syrup, cellulose derivatives or hydrogenated edible fats or oils); emulsifying agents (e.g., lecithin or acacia); non-aqueous vehicles (e.g., almond oil, oily esters, or fractionated vegetable oils); and preservatives (e.g., methyl or propyl-p-hydroxybenzoates or sorbic acid). The pharmaceutical compositions may take the form of, for example, tablets, capsules or pellets prepared by conventional means with pharmaceutically acceptable excipients such as binding agents (e.g., pregelatinized maize starch, polyvinyl pyrrolidone or hydroxypropyl methylcellulose); fillers (e.g., lactose, microcrystalline cellulose or calcium hydrogen phosphate); lubricants (e.g., magnesium stearate, talc or silica); disintegrants (e.g., potato starch or sodium starch glycolate); or wetting agents (e.g., sodium lauryl sulphate). The tablets may be coated by methods well-known in the art.

For buccal administration, the compositions may take the form of tablets, troche or lozenge formulated in conventional manner. Compositions for oral or buccal administration, may be formulated to give controlled release of the active compound. Such formulations may include one or more sustained-release agents known in the art, such as glyceryl mono-stearate, glyceryl distearate and wax.

Compositions may be applied topically. Such administrations include applying the compositions externally to the epidermis, the mouth cavity, eye, ear and nose. This contrasts with systemic administration achieved by oral, intravenous, intraperitoneal and intramuscular delivery.

Compositions for use in topical administration include, e.g., liquid or gel preparations suitable for penetration through the skin such as creams, liniments, lotions, ointments or pastes, and drops suitable for delivery to the eye, ear or nose.

In some embodiments, the present compositions include creams, drops, liniments, lotions, ointments and pastes are liquid or semi-solid compositions for external application. Such compositions may be prepared by mixing the active ingredient(s) in powdered form, alone or in solution or suspension in an aqueous or non-aqueous fluid with a greasy or non-greasy base. The base may comprise complex hydrocarbons such as glycerol, various forms of paraffin, beeswax; a mucilage; a mineral or edible oil or fatty acids; or a macrogel. Such compositions may additionally comprise suitable surface active agents such as surfactants, and suspending agents such as agar, vegetable gums, cellulose derivatives, and other ingredients such as preservatives, antioxidants, and the like.

Further, the present composition can be administered nasally or by inhalation. For nasal or inhalation administration, the compositions are conveniently delivered in the form of an aerosol spray presentation from pressurized packs or a nebulizer, with the use of a suitable propellant, e.g., dichlorodifluoromethane, trichlorofluoromethane, dichlorotetrafluoroethane, carbon dioxide or other suitable gas. In the case of a pressurized aerosol the dosage unit may be determined by providing a valve to deliver a metered amount. Capsules and cartridges of, e.g., gelatin for use in an inhaler or insufflator may be formulated containing a powder mix of the compound and a suitable powder base such as lactose or starch.

Some of the present compositions can be formulated as a depot preparation. Such long acting formulations may be administered by implantation (for example, subcutaneously or intramuscularly) or by intramuscular injection. Thus, for example, the compounds may be formulated with suitable polymeric or hydrophobic materials (for example, as an emulsion in an acceptable oil) or ion exchange resins, or as sparingly soluble derivatives, for example, as a sparingly soluble salt. Liposomes and emulsions are well known examples of delivery vehicles or carriers for hydrophilic drugs.

Techniques and formulations for administering above-described compositions may be found in Remington's Pharmaceutical Sciences, Meade Publishing Col., Easton, Pa., latest edition.

The effectiveness of any of the aforementioned hydroxylamines and derivatives thereof in inhibiting angiogenesis may be determined by one of several accepted biological assays as known in the art. One preferred method is the chick chorioallantoic membrane (CAM) assay. In the CAM bioassay, fertilized chick embryos are cultured in Petri dishes. On day 6 of development, a disc of a release polymer, such as methyl cellulose, impregnated with the test sample or an appropriate control substance is placed onto the vascular membrane at its advancing edge. On day 8 of development, the area around the implant is observed and evaluated. Avascular zones surrounding the test implant indicate the presence of an inhibitor of embryonic neovascularization. Moses et al., 1990, Science, 248:1408-1410 and Taylor et al., 1982, Nature, 297:307-312. The reported doses for previously described angiogenesis inhibitors tested alone in the CAM assay are 50 μg of protamine (Taylor et al. (1982)), 200 μg of bovine vitreous extract (Lutty et al., 1983, Invest. Opthalmol. Vis. Sci. 24:53-56), and 10 μg of platelet factor IV (Taylor et al. (1982)). The lowest reported doses of angiogenesis inhibitors effective as combinations include heparin (50 μg) and hydrocortisone (60 μg), and B-cyclodextrin tetradecasulfate (14 μg) and hydrocortisone (60 μg), reported by Folkman et al., 1989, Science 243:1490.

The following examples are provided to describe the invention in greater detail. They are intended to illustrate, not to limit, the invention.

### EXAMPLE 1

### Chick Chorioallantoic Membrane (CAM) Model for Angiogenesis Studies

Neovascularization was examined by previously described methods (see references at the end of Example 5). Ten-day-old fertilized chicken eggs were incubated at 37° C. with 55% relative humidity. In the dark with the help of candling lamp and using a hypodermic needle a small hole was punctured in the shell covering the air sac. A second hole was punctured on the wider side of the egg above an avascular area of the embryonic membrane. An artificial air sac was created below the second hole by applying gentle vacuum to the first hole using a small rubber squeeze bulb. The vacuum caused the separation of chorioallantoic membrane (CAM) from the shell. An approximately 1.0 cm² widow was cut in the shell over the dropped CAM with the use of a mini drill. The underlying CAM was accessed through this small window.

Filter disks were punched using a small puncher from filter paper #1 (Whatman International, United Kingdom). Filter disks were soaked in 3 mg/ml cortisone acetate solution (95% ethanol and water) and air-dried under sterile condition. For inducing angiogenesis, sterile filter disks were saturated with bFGF (1 μg/ml) or other pro-angiogenesis factors and control disks were saturated with PBS without Calcium and Magnesium.

Using sterile forceps one filter/CAM was placed from the window. The window was sealed with Highland brand transparent tape. After 24 hr, 10-25 □l of test agent (inhibitor) was injected intravenously or added topically into the CAM membrane of bFGF or other pro-angiogenesis factors stimulated CAMs.
Control filter disks received PBS without Calcium and Magnesium.
After 48 hr, CAM tissue directly beneath filter disk was harvested and placed in a 35-mm Petri dish. Eight-Ten eggs/treatment group was used.

A pro-angiogenic agent (see Examples, below) was added to induce new blood vessel branches on the CAM of 10-day old embryos. Sterile disks of #1 filter paper (Whatman International, United Kingdom) were pre-treated with 3 mg/ml cortisone acetate, and air dried under sterile conditions. The disks were then suspended in PBS (Phosphate Buffered Saline) and placed on growing CAMs. Filters treated with TPH (TEMPOL-H) or TEMPOL and/or H₂O₂ or TPH and/or bFGF or VEGF were placed on the first day of the 3-day incubation.

Digital Images and Microscopic analysis of CAM sections: CAM sections from Petri dish were examined using SV6 stereomicroscope (Karl Zeiss) at 50× magnification. Digital images were captured using a 3-CCD color video camera system (Toshiba America, New York, N.Y.). These images were analyzed using Image-Pro Plus software (Media Cybernetics). The number of branch points in blood vessels within the circular region superimposed to the area of a filter disk was counted for each section. After incubation at 37° C. with 55% relative humidity for 3 days, the CAM tissue directly beneath each filter disk was resected from control and treated CAM samples. Tissues were washed three times with PBS. Sections were placed in a 35-mm Petri dish (Nalge Nunc; Rochester, N.Y.) and were examined under a SV6 stereomicroscope (Karl Zeiss; Thornwood, N.Y.) at 50× magnification. Digital images of CAM sections adjacent to filters were collected using a 3-CCD color video camera system (Toshiba America; New York, N.Y.) and analyzed with the Image-Pro Plus software (Media Cybernetics; Silver Spring, Md.). The number of vessel branch points contained in a circular region equal to the area of a filter disk was counted for each section. Percent inhibition data are expressed as the quotient of the experimental value minus the negative control value divided by the difference between the positive control value and the negative control value. One image was counted in each CAM preparation, and findings from eight CAM preparations were analyzed for each treatment condition. In addition, each experiment was performed three times. The resulting angiogenesis index is the mean±SEM (Standard Error of Measurement) of new branch points in each set of treatment.

Statistical Analysis: Statistical analysis of blood vessel branching patterns are performed by 1-way analysis of variance (ANOVA) comparing experimental with corresponding control groups. Statistical significance differences are assessed at P value of <0.05.

### EXAMPLE 2

### Effect of TPH and Tempol on Angiogenesis Induced by H₂O₂

TPH (TEMPOL-H, the hydroxylamine reduced form of the nitroxide 4-hydroxy-2,2,6,6-tetramethylpiperidin-1-yloxy) or TEMPOL (4-hydroxy-2,2,6,6-tetramethylpiperidine-N-oxyl radical) was applied to the CAM model study to determine its respective anti-angiogenesis effects according to the materials and methods provided in Example 1. H₂O₂ was used to induce angiogensis in the CAM model. The CAM model study produced the results shown in Tables 1A and 1B.

TABLE 1A
Anti-angiogenesis efficacy of TPH versus TEMPOL at
100-200 μg in H₂O₂-induced angiogenesis in the CAM model

| | Branch | % |
|---|---|---|
| Treatment | pts ± SEM | Inhibition ± SD |
| PBS | 82.9 ± 4.8 | |
| H₂O₂ (88 μM, 30 ng) | 185.0 ± 17.0 | |
| H₂O₂ (88 μM, 30 ng) + | 152.2 ± 18.8 | 32.1 ± 14.1* |
| TEMPOL (100 μg) | | |
| H₂O₂ (88 μM, 30 ng) + | 151.8 ± 12.5 | 32.5 ± 12.1* |
| TEMPOL (200 μg) | | |
| H₂O₂ (88 μM, 30 ng) + | 155 ± 14.4 | 28.0 ± 13.6* |
| TPH (100 μg) | | |
| H₂O₂ (88 μM, 30 ng) + | 151.3 ± 15.3 | 32.9 ± 8.4* |
| TPH (200 μg) | | |

Data represent mean ± SD, n = 8 per group,
*P < 0.05 as compared to H₂O₂.

TABLE 1B
Anti-angiogenesis efficacy of TPH versus TEMPOL at
400-800 μg in H₂O₂-induced angiogenesis in the CAM model

| | Branch | % |
|---|---|---|
| Treatment | pts ± SEM | Inhibition ± SD |
| PBS | 88.0 ± 8.9 | |
| H₂O₂ (88 μM, 30 ng) | 177.0 ± 9.5 | |
| H₂O₂ (88 μM, 30 ng) + | 150.0 ± 7.7 | 30.1 ± 8.7* |
| TEMPOL(400 μg) | | |
| H₂O₂ (88 μM, 30 ng) + | 122.8 ± 3.0 | 60.9 ± 3.2** |
| TEMPOL(800 μg) | | |
| H₂O₂ (88 μM, 30 ng) + | 137.2 ± 6.9 | 44.7 ± 6.6** |
| TPH(400 μg) | | |
| H₂O₂ (88 μM, 30 ng) + | 127.3 ± 6.4 | 55.7 ± 7.2** |
| TPH(800 μg) | | |

Data represent mean ± SD, n = 8 per group,
*P < 0.05 and
**P < 0.01 as compared to H₂O₂.

As can be seen from the tables, either TPH or TEMPOL effectively inhibited angiogenesis-induced by super-maximal concentrations of H₂O₂ in the CAM model.

### EXAMPLE 3

### Effect of TPH on bFGF-Induced Angiogenesis

TPH was applied to the CAM model study to determine its respective anti-angiogenesis effects according to the materials and methods provided in Example 1. Basic Fibroblast Growth Factor (bFGF) was used to induce angiogenesis in the CAM model. The CAM model study produced the results shown in Table 2.

TABLE 2
Anti-angiogenesis efficacy of TPH in inhibiting
bFGF-induced angiogenesis in the CAM model

| | Mean Branch | Mean % |
|---|---|---|
| Treatment | points ± SEM | Inhibition ± SD |
| PBS | 92.8 ± 12.5 | |
| bFGF(1 μg/ml) | 192.2 ± 7.6 | |
| bFGF(1 μg/ml) + | 172 ± 12.4 | 20 ± 12 |
| TPH (100 μg) 4-1 | | |
| bFGF(1 μg/ml) + | 147.2 ± 7.5 | 45.3 ± 7.5** |
| TPH (200 μg) 3-1 | | |
| bFGF(1 μg/ml) + | 133.8 ± 10.8 | 58.7 ± 10.9** |
| TPH (400 μg) 2-1 | | |
| bFGF(1 μg/ml) + | 164.2 ± 6.7 | 28.1 ± 6.8* |
| TPH (800 μg) 1-1 | | |

Data represent mean ± SD, n = 8 per group,
*P < 0.05 and
**P < 0.01 as compared to bFGF.

TPH resulted in dose-dependent inhibition (100-400 μg) of bFGF-induced angiogenesis in the CAM model (Table 2).

### EXAMPLE 4

### Effect of TPH on VEGF-Induced Angiogenesis

TPH was applied to the CAM model study to determine its respective anti-angiogenesis effects according to the materials and methods provided in Example 1. VEGF was used to induce angiogenesis in the CAM model. Results are shown in Table 3.

TABLE 3
Anti-angiogenesis efficacy of TPH in inhibiting
VEGF-induced angiogenesis in the CAM model

| | Mean Branch | Mean % |
|---|---|---|
| Treatment | points ± SD | Inhibition ± SD |
| PBS | 87.0 ± 9.6 | |
| VEGF (2 μg/ml) | 195.7 ± 12.1 | |
| VEGF + TPH(100 μg) | 154.8 ± 7.5 | 37.6 ± 6.9** |
| VEGF + TPH(200 μg) | 150 ± 3.8 | 42 ± 3.5** |
| VEGF + TPH(400 μg) | 137.8 ± 3.0 | 53.3 ± 3.3** |
| VEGF + TPH(800 μg) | 118.8 ± 9.9 | 70.7 ± 9.1** |

Data represent mean ± SD, n = 8 per group,
**P < 0.01 as compared to VEGF.

TPH demonstrated dose-dependent inhibition of VEGF-induced angiogenesis in the CAM model (Table 3). The anti-angiogenesis efficacy of TPH was much greater against VEGF-induced angiogenesis as compared with that observed against bFGF (Tables 2 and 3).

### EXAMPLE 5

### Effect of Injected Compound 1 (Cyclopropanecarboxylic acid 1-hydroxy-2,2,6,6-tetramethyl-piperidin-4-yl ester) in bFGF-stimulated CAM Model

Compound 1 was introduced via injection to the CAM model study to determine its respective anti-angiogenesis effects according to the materials and methods provided in Example 1. bFGF was used to induce angiogenesis in the CAM model. Results are shown in Table 4.

TABLE 4

| | Branch | % |
|---|---|---|
| Treatment | pts ± SEM | Inhibition ± SEM |
| bFGF (1 ug) + PBS | 147 ± 7.5 | |
| bFGF (1 ug) + PBS injected | 139 ± 8 | 9 ± 5 |
| bFGF (1 ug) + OT-551 (30 ug) | 87 ± 3 | 74 ± 4 |
| injected | | |

### REFERENCES FOR EXAMPLES 1-5

1. Powell, J. A., Mohamed, S., Kerr, J., Mousa, S. A.: J. Cellular Biochemistry 80: 104-114; 2000.
2. Auerbach R, Kubai L, Knighton D, Folkman J. Dev Biol. 1974;41 :391-394.
3. Marcinkiewicz C, Weinreb, P H, Calvete, J J, Kisiel, D G, Mousa, S A, Tuszynski, G P, Lobb, R R. Obtustatin: Cancer Res. 63(9): 2020-2023; 2003.
4. Colman, R W, Pixley, R A, Sainz, I M, Song, J S, Isordia-Salas, Mohamed S, Powell, J, Mousa, S A: J Thrombosis Haemostasis 1 (1); 164-173; 2003.
5. Dupont E, Falardeau P, Mousa S A, Dimitriadou V, Pepin M C, Wang T, Alaoui-Jamali M A. Clin Exp Metastasis. 2002;19:145-153.
6. Kim S, Bell K, Mousa S A, Varner J A Am J Path. 2000;156:1345-1362.
7. Mousa S A, Mousa A S. Current Pharmaceutical Design 2004; 10(1): 1-9.
8. Polytarchou C and Papadimitriou E: Free Radical Research 38 (5): 501-508.
9. Mousa S A, O'Connor L, Davis F B, Davis P J. Proangiogenesis action of the thyroid hormone analog 3,5-diiodothyropropionic acid (DITPA) is initiated at the cell surface and is integrin mediated. Endocrinology. 2006; 147(4):1602-7.
10. Mousa S A: Alpha v integrin affinity/specificity and anti-angiogenesis effect of a novel tetraaza cyclic peptide derivative, SU015, in various species. J Cardiovascular Pharmacology 2005; 45(5): 462-467.
11. Davis F B, Mousa S A, O'Connor L, Mohamed S, Lin H Y, Cao H J, Davis P J. Proangiogenic action of thyroid hormone is fibroblast growth factor-dependent and is initiated at the cell surface. Circulation Research 2004; 94(11):1500-1506.
12. Mousa S A, Mohamed S, Wexler E J, Kerr J S. Anti-angiogenesis and anticancer efficacy of TA138, a novel alphavbeta3 antagonist. Anticancer Res. 2005; 25(1A): 197-206.
13. Cezary Marcinkiewicz, Paul H. Weinreb, Juan J. Calvete, Dariusz G. Kisiel, Shaker A. Mousa, George P. Tuszynski, Roy R. LobbL: Obtustatin, a potent selective inhibitor of alpha1/Beta1 integrin in vitro and angiogenesis in vivo. Cancer Research 63: 2020-2023, 2003.
14. Colman, R. W., Pixley, R. A., Sainz, I. M., Song, J. S., Isordia-Salas, Mohamed S., Powell, J., Mousa, S. A.: Inhibition of angiogenesis by antibody blocking the action of proangiogenic high-molecular-weight kininogen. J Thrombosis Haemostasis 1 (1); 164-173, 2003.
15. Dupont, E., Falardeau, P., Mousa, S. A., Dimitriadou, V., Pepin, M. C., Wang, T., Alaoui-Jamali, M. A.: Antiangiogenic and antimetastatic properties of Neovastat (AE-941), an orally active extract. Clin Exp Metastasis 19(2):145-153, 2002.

### EXAMPLE 6

### In Vitro Stability Analysis of Compound 1 in Rat, Rabbit, Dog, and Human Plasma

The active metabolite of Compound 1 (Cyclopropanecarboxylic acid 1-hydroxy-2,2,6,6-tetramethyl-piperidin-4-yl ester) is TPH. The objective of this analysis was to determine the in vitro half-life of Compound 1 in rat, rabbit, dog, and human plasma under standardized incubation conditions.

Compound 1 was incubated with pooled rat, rabbit, dog, and human plasma for various times under standardized incubation conditions. Pre-labeled tubes containing pooled plasma from rats, rabbits, dogs, and humans were pre-incubated in a shaking 37° C. water bath. A Compound 1 solution was added to the tubes at a final concentration of 1000 ng/mL. Time zero samples (n=5) were immediately removed and transferred into tubes containing a stabilizer solution (DTPA, acetylcysteine and ascorbic acid), the LC/MS/MS assay internal standard and methanol. The stabilizer solution has been demonstrated to stabilize Compound 1 in the presence of plasma from rats, rabbits, dogs, and humans. The tubes were vortexed, placed on ice, followed by centrifugation. One hundred-μL aliquots of the supernatant were transferred into HPLC sample vials. Additional tubes (n=5 at each time point) were incubated for 5, 10, 20, 30, 60, 120, and 240 minutes at 37° C. and thereafter processed. The amount of Compound 1 and TPH in each incubated sample was quantified using validated LC/MS/MS assays.

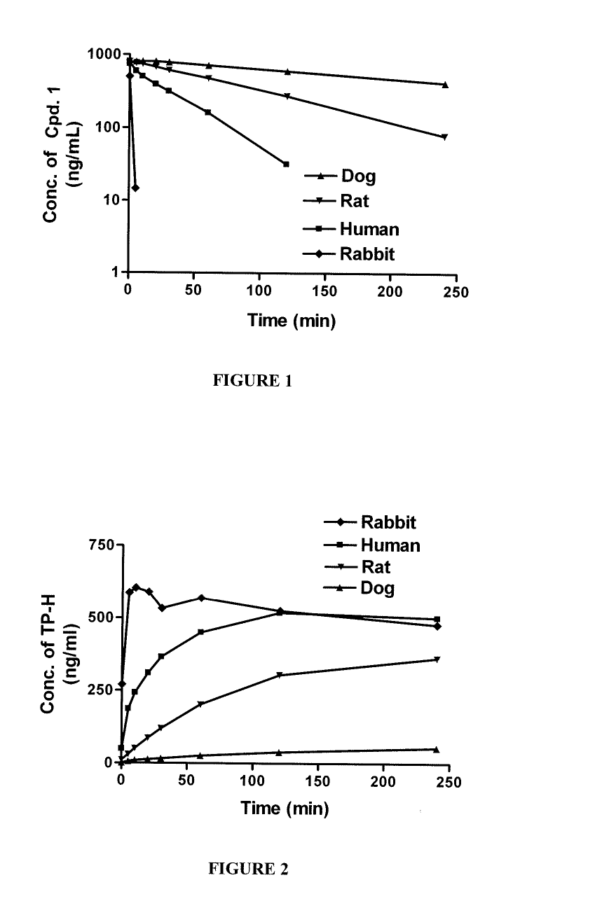

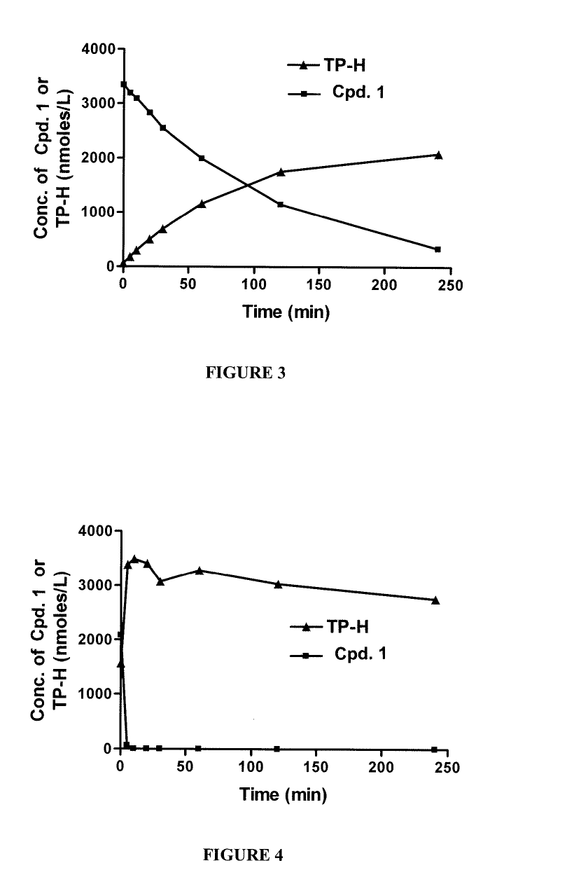

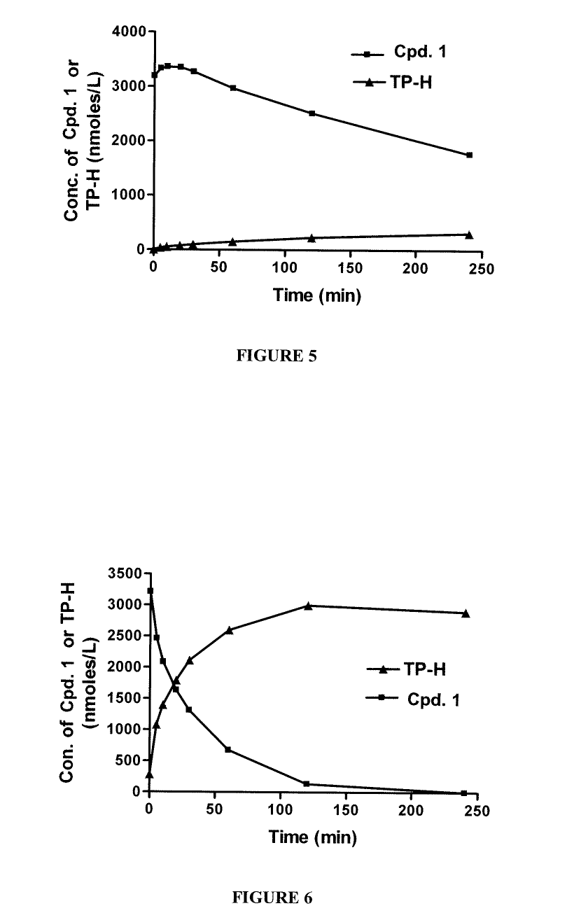

The disappearance of Compound 1 and appearance of TPH as a function of incubation time with rat, rabbit, dog, and human plasma are summarized in Tables 5 and 6, respectively and shown in FIGS. 1 and 2, respectively. FIGS. 3 to 6 show the temporal relationships between the disappearance of Compound 1 and the appearance of TPH in rat, rabbit, dog, and human plasma.

TABLE 5
Concentrations (ng/mL) of Compound 1^{a} in Rat, Rabbit, Dog, and Human Plasma
as a Function of Incubation Time Under Standardized Incubation Conditions

| Time (min) | Rat | Rabbit | Dog | Human |
|---|---|---|---|---|
| 0 | 806.50 ± 86.77 | 502.75 ± 74.66 | 771.12 ± 21.68 | 775.47 ± 22.50 |
| 5 | 770.32 ± 20.66 | 14.56 ± 3.35 | 804.93 ± 17.45 | 593.43 ± 7.55 |
| 10 | 745.77 ± 15.50 | 0.00 ± 0.00 | 811.14 ± 21.06 | 503.18 ± 20.90 |
| 20 | 682.88 ± 16.94 | 0.00 ± 0.00 | 809.01 ± 18.58 | 394.69 ± 6.72 |
| 30 | 613.79 ± 25.84 | 0.00 ± 0.00 | 789.53 ± 13.73 | 316.37 ± 7.67 |
| 60 | 480.48 ± 10.69 | 0.00 ± 0.00 | 717.22 ± 25.73 | 162.41 ± 9.77 |
| 120 | 277.94 ± 5.55 | 0.00 ± 0.00 | 608.14 ± 25.96 | 32.22 ± 2.63 |
| 240 | 80.70 ± 2.02 | 0.00 ± 0.00 | 428.20 ± 12.03 | 0.00 ± 0.00 |
| T½ (min) | 69.54 | 0.98 | 239.60 | 27.78 |

Data are expressed as mean ± SD (n = 5)
^{a}Cyclopropanecarboxylic acid 1-hydroxy-2,2,6,6-tetramethyl-piperidin-4-yl ester

TABLE 6
Concentrations (ng/mL) of TPH in Rat, Rabbit, Dog, and Human Plasma as a Function
of OT-551 Incubation Time Under Standardized Incubation Conditions

| Time (min) | Rat | Rabbit | Dog | Human |
|---|---|---|---|---|
| 0 | 10.23 ± 1.59 | 270.55 ± 35.45 | 0.00 ± 0.00 | 48.76 ± 2.84 |
| 5 | 30.47 ± 1.65 | 587.17 ± 21.99 | 5.85 ± 0.73 | 186.71 ± 4.58 |
| 10 | 50.91 ± 2.11 | 604.21 ± 21.99 | 8.77 ± 0.65 | 241.99 ± 8.05 |
| 20 | 86.30 ± 3.30 | 590.06 ± 40.97 | 12.79 ± 0.74 | 310.10 ± 8.54 |
| 30 | 119.63 ± 7.08 | 533.01 ± 117.40 | 16.22 ± 0.67 | 365.53 ± 14.44 |
| 60 | 201.94 ± 4.19 | 569.19 ± 32.96 | 25.68 ± 1.04 | 449.85 ± 9.73 |
| 120 | 304.66 ± 7.27 | 525.63 ± 10.31 | 39.31 ± 1.09 | 519.12 ± 19.52 |
| 240 | 362.66 ± 7.50 | 477.54 ± 40.95 | 53.92 ± 1.68 | 501.39 ± 11.33 |

Data are expressed as mean ± SD (n = 5)

The hydrolysis rate of Compound 1 (Cyclopropanecarboxylic acid 1-hydroxy-2,2,6,6-tetramethyl-piperidin-4-yl ester) differed across species. Compound 1 was fairly stable in dog plasma, with an in vitro half-life averaging 4 hours. In contrast, the compound was hydrolyzed rapidly in rabbit plasma with an in vitro half-life averaging only 1 minute. Esterases in human and rat plasma were intermediate in activity. The in vitro half-life of Compound 1 averaged 28 minutes and 70 minutes in human and rat plasma, respectively.

The disappearance of Compound 1 coincided with the formation of TPH. Within experimental limits, the disappearance of Compound 1 in the incubation mixture can be accounted for on a molar basis by the formation of TPH. These results suggested that under the standardized incubation conditions, hydrolysis of the ester functionality in Compound 1 forming TPH was the primary pathway of Compound 1 metabolism and TPH was stable during the 240 minute incubation period.

### EXAMPLE 7

### Single-Dose Intravenous Toxicity Analysis of Compound 1 HCl Administered to Sprague-Dawley Rats

The objective of this analysis was to determine the toxicokinetic parameters of Compound 1 and the active metabolite, TPH, as part of a single 10-minute intravenous infusion toxicity analysis of Compound 1 in Sprague-Dawley rats.

Compound 1 was administered once to each animal via an intravenous infusion into a lateral tail vein at a dose level of 0 (saline), 10, 30, 100, or 200 mg/kg (30 mL/kg over 10 minutes). Blood for toxicokinetic evaluations was collected at pre-determined time points during and after the infusion. Plasma samples were analyzed for Compound 1 and TPH using validated LC/MS/MS assays.

Descriptive toxicokinetic parameters were determined by standard model independent methods (Gilbaldi and Perrier, 1982) based on the plasma concentration-time data. All pharmacokinetic analyses were performed using Kinetica®, version 4.2 (Innaphase, Philadelphia, Pa.).

Cₘₐₓ is the observed maximum plasma concentration

Tₘₐₓ is the time Cₘₐₓ is reached

AUC(0-4.167 hr) is the area under the plasma concentration-time curve from the start of the 10 minute infusion to 4 hours after the termination of the infusion

AUC is the area of the plasma concentration-time curve from the start of the 10-minute infusion to time infinity

T_{1/2} is the elimination half-life

The plasma concentrations were rounded to the nearest tenth of a ng/mL before the calculations. Plasma samples with concentrations below the quantifiable assay limit (<50 ng/mL for Compound 1 and <20 ng/mL for TPH) were assigned a value of zero for pharmacokinetic analyses and generation of means and SD. Nominal time points were used for all calculations.

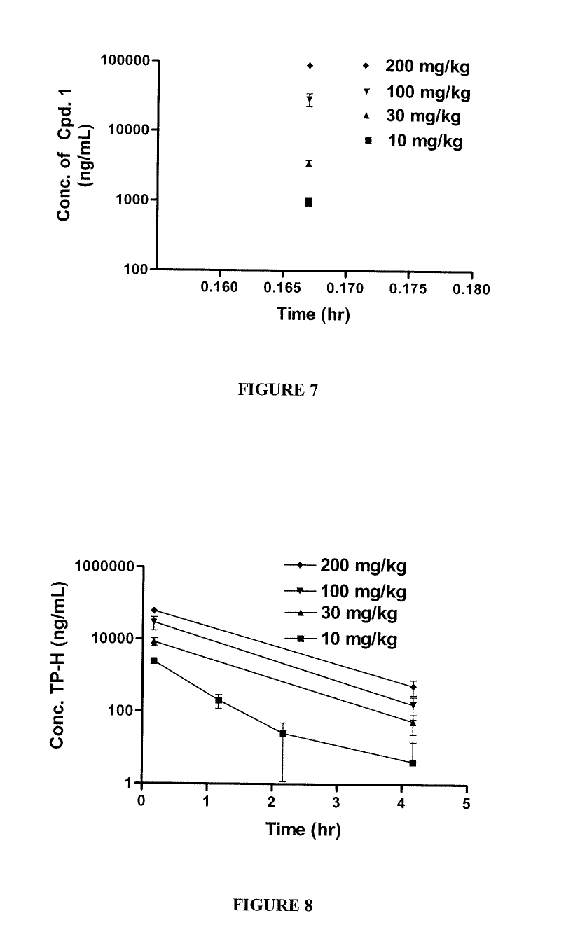

Since there was no apparent gender difference in the plasma concentrations of Compound 1 and TPH, the data for male and female rats at each sampling time point were pooled. The mean concentrations of Compound 1 and TPH at the end of the 10-minute intravenous infusion and several time points after termination of the infusion are summarized in Tables 7 and 8, respectively. FIGS. 7 and 8 show the plasma concentration-time profiles of Compound 1 and TPH, respectively.

TABLE 7
Mean ± SD Plasma Concentrations (ng/mL) and Toxicokinetic Parameters of Compound 1 in
Sprague-Dawley Rats (n = 5-6) After a Single 10-Minute Intravenous Infusion of Compound 1

| | Time | Dose (mg/kg) | | | | |
|---|---|---|---|---|---|---|
| Parameters | (hr)^{a} | 0 | 10 | 30^{b} | 100 | 200 |
| | 0.167 | 0.0 | 980.5 ± 310.6 | 3487.1 ± 808.9 | 29020.0 ± 15106.5 | 89740.8 ± 18142.1 |
| | 1.167 | NS | 0.0 | NS | NS | NS |
| | 2.167 | NS | 0.0 | NS | NS | NS |
| | 4.167 | 0.0 | 0.0 | 0.0 | 0.0 | 0.0 |
| Cmax (ng/mL) | | NA | 980.5 | 3487.1 | 29020.0 | 89740.8 |
| Tmax (hr) | | NA | 0.167 | 0.167 | 0.167 | 0.167 |

^{a}Timing relative to the start of the intravenous infusion;
^{b}n = 5
NS: No Sample

TABLE 8
Mean ± SD Plasma Concentrations (ng/mL) and Toxicokinetic Parameters of TPH in Sprague-Dawley
Rats (n = 5-6) After a Single 10-Minute Intravenous Infusion of Compound 1

| | Time | Dose (mg/kg) | | | | |
|---|---|---|---|---|---|---|
| Parameters | (hr)^{a} | 0 | 10 | 30^{b} | 100 | 200 |
| | 0.167 | 0.0 | 2481.7 ± 325.8 | 8337.7 ± 2099.5 | 29020.8 ± 11713.7 | 60802.2 ± 8922.5 |
| | 1.167 | NS | 204.7 ± 85.6 | NS | NS | NS |
| | 2.167 | NS | 25.2 ± 24.0 | NS | NS | NS |
| | 4.167 | 0.0 | 4.2 ± 10.3 | 53.8 ± 29.7 | 160.0 ± 97.5 | 524.5 ± 237.0 |
| Cmax (ng/mL) | | NA | 2481.7 | 8337.7 | 29020.8 | 60802.2 |
| Tmax (hr) | | NA | 0.167 | 0.167 | 0.167 | 0.167 |
| AUC_{(0-4.167 hr)} (ng/mL · hr) | | NA | 1694.8 | NA | NA | NA |
| AUC (ng/mL · hr) | | NA | 1697.5 | NA | NA | NA |
| T½ (hr) | | NA | 0.4 | NA | NA | NA |

^{a}Timing relative to the start of the 10-minute intravenous infusion;
^{b}n = 5
NS: No Sample;
NA: Not Applicable

Dose-related increases in plasma levels of Compound 1 were observed immediately after termination of the 10-minute infusion over the dosage range of 10 to 200 mg/kg. The peak concentrations at the end of the infusion averaged 980.5, 3487.1, 29020.0 and 89740.8 ng/mL after 10, 30, 100, and 200 mg/kg, respectively. Compound 1 was not quantifiable at one hour after termination of the infusion after 10 mg/kg. At the three higher dosages of 30 to 200 mg/kg, plasma levels of Compound 1 in samples collected at four hours after termination of the infusion were not quantifiable. The elimination half-life of Compound 1 was not determinable based on the available data but the results suggested that the clearance of Compound 1 in rats was very rapid.

Dose-related increases in plasma levels of TPH were also observed immediately after termination of the 10-minute infusion of Compound 1. The peak concentrations were observed at the end of the Compound 1 infusion and averaged 2481.7, 8337.7, 29020.8, and 60802.1 ng/mL after 10, 30, 100, and 200 mg/kg, respectively. Similar to Compound 1, plasma levels of TPH decreased rapidly at the end of the infusion of Compound 1 but were still quantifiable at 4 hr post infusion of a 10 mg/kg dose. The terminal elimination half-life of TPH after the 10 mg/kg dose was estimated to be 0.4 hr. The elimination half-life of TPH after 30, 100 and 200 mg/kg was not determinable based on the available data but plasma samples collected at four hours after terminating the infusion of the three higher Compound 1 doses indicated that levels of TPH were less than 1% of the concentrations observed immediately after terminating the infusions of Compound 1.

### EXAMPLE 8

### Anti-Angiogenesis Efficacy and Mechanism(s) of TPH in a Human Endothelial 3-Dimensional Sprouting Model

The protocol set forth below is performed to determine the anti-angiogenesis efficacy of TPH in a 3-D sprouting assay using human endothelial cells (micro-vascular, retinal, and choriodal endothelial cells), and further to determine the anti-angiogenesis efficacy in response to oxidative stress, b-FGF, VEGF, TNF-alpha, monocytes, and lipopolysaccharide (LPS).

Experimental Design:

Three-Dimensional Angiogenesis Assay: In Vitro 3D Sprout Angiogenesis of Human Dermal Micro-vascular Endothelial Cells (HDMEC) Cultured on micro-carrier beads coated with fibrin: Confluent HDMEC (passages 5-10) are mixed with gelatin-coated Cytodex-3 beads with a ratio of 40 cells per bead. Cells and beads (150-200 beads per well for 24-well plate) are suspended with 5 ml Endothelial Basal Medium (EBM)+15% normal human serum (HS), mixed gently every hour for first 4 hours, then left to culture in a CO₂ incubator overnight. The next day, 10 ml of fresh EBM+5% HS are added, and the mixture is cultured for another 3 hours. Before experiments, the culture of EC-beads is checked, then, 500 μl of phosphate-buffered saline (PBS) is added to a well of 24-well plate, and 100 μl of the EC-bead culture solution is added to the PBS. The number of beads is counted, and the concentration of EC/beads is calculated.

A fibrinogen solution (1 mg/ml) in EBM medium, with or without angiogenesis factors or testing factors, is prepared. For positive control, 30 ng/ml VEGF+25 ng/ml FGF2 is used. EC-beads are washed with EBM medium twice, and EC-beads are added to fibrinogen solution. The experiment is done in triplicate for each condition. The EC-beads are mixed gently in fibrinogen solution, and 2.5 μl human thrombin (0.05 U/μl) is added in 1 ml fibrinogen solution; 300 μl is immediately transferred to each well of a 24-well plate. The fibrinogen solution polymerizes in 5-10 minutes; after 20 minutes, EBM+20% normal human serum+10 μg/ml Aprotinin is added, and the plate is incubated in a CO₂ incubator. It takes about 24-48 hours for HDMEC to invade fibrin gel and form tubes.

A micro-carrier in vitro angiogenesis assay previously designed to investigate bovine pulmonary artery endothelial cell angiogenic behavior in bovine fibrin gels (Nehls & Drenkhahn, 1995, Microvascular Research 50: 311-322; Nehls & Drenkhahn, 1995, Histochem. & Cell. Biol. 104: 459-466) is modified for the study of human microvascular endothelial cell angiogenesis in three-dimensional ECM (Extra Cellular matrix) environments. Briefly, human fibrinogen, isolated as previously described (Feng et al., 1999, J. Invest. Dermatol. 113: 913-919; Mousa et al., 2005, Endocrinology Dec. 29, 2005: 1390), is dissolved in M199 medium at a concentration of 1 mg/ml (pH 7.4) and sterilized by filtering through a 0.22 micron filter. An isotonic 1.5 mg/ml collagen solution is prepared by mixing sterile Vitrogen 100 in 5× M199 medium and distilled water. The pH is adjusted to 7.4 by 1N NaOH. In certain experiments, growth factors and ECM proteins (such as VEGF, bFGF, PDGF (Platelet-Derived Growth Factor), serum, gelatin, and fibronectin) are added to the fibrinogen or collagen solutions. About 500 EC-beads are then added to the 1 mg/ml fibrinogen or 1.5 mg/ml collagen solutions. Subsequently, EC-beads-collagen or EC-beads-fibrinogen suspension (500 EC-beads/ml) is plated onto 24-well plates at 300 μl/well. EC-bead-collagen cultures are incubated at 37° C. to form gel. The gelling of EC-bead-fibrin cultures occurrs in less than 5 minutes at room temperature after the addition of thrombin to a final concentration of 0.5 U/ml. After gelation, 1 ml of fresh assay medium (EBM supplemented with 20% normal human serum for HDMEC or EBM supplemented with 10% fetal bovine serum for BAEC (Bovine Aortic Endothelial Cells)) is added to each well. The angiogenic response is monitored visually and recorded by video image capture. Specifically, capillary sprout formation is observed and recorded with a Nikon Diaphot-TMD inverted microscope (Nikon Inc.; Melville, N.Y.), equipped with an incubator housing with a Nikon NP-2 thermostat and Sheldon #2004 carbon dioxide flow mixer. The microscope is directly interfaced to a video system consisting of a Dage-MTI CCD-72S video camera and Sony 12″ PVM-122 video monitor linked to a Macintosh G3 computer. The images are captured at various magnifications using Adobe Photoshop. The effect of angiogenic factors on sprout angiogenesis is quantified visually by determining the number and percent of EC-beads with capillary sprouts. One hundred beads (five to six random low power fields) in each of triplicate wells are counted for each experimental condition. All experiments are repeated at least three times. Statistical analysis is performed by one-way analysis of variance comparing experimental with respective control group and statistical significance is calculated based on P<0.05.

While the present invention has been particularly shown and described with reference to the presently preferred embodiments, it is understood that the invention is not limited to the embodiments specifically disclosed and exemplified herein. Numerous changes and modifications may be made to the preferred embodiments of the invention without departing from the scope and spirit of the invention as set forth in the appended claims.

What is claimed:

1. A method of inhibiting angiogenesis in a patient, comprising:
administering to the patient a hydroxylamine compound or an ester derivative thereof, wherein the ester derivative comprises formula I, in a therapeutically sufficient amount to inhibit the angiogenesis;
wherein formula I is: 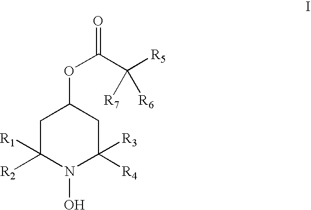
wherein R₁ and R₂ are, independently, H or C₁ to C₃ alkyl;
R₃ and R₄ are, independently, C₁ to C₃ alkyl;
R₅ is H, OH, or C₁ to C₆ alkyl;
R₆ is or C₁ to C₆ alkyl, alkenyl, alkynyl, or substituted alkyl or alkenyl;
R₇ is C₁ to C₆ alkyl, alkenyl, alkynyl, or substituted alkyl or alkenyl;
wherein R₁ and R₂, taken together, or R₃ and R₄, taken together, or both are cycloalkyl;
wherein R₆ and R₇, or R₅, R₆ and R₇, taken together, form a carbocycle or heterocycle comprising a 3 to 7 membered ring.

2. The method of claim 1, wherein the hydroxylamine compound is:

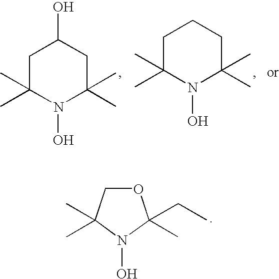

3. The method of claim 1, wherein the hydroxylamine compound is:

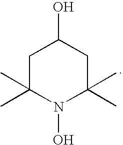

4. The method of claim 1 wherein R₁, R₂, R₃, and R₄ are a C₁-C₃ alkyl.

5. The method of claim 1 wherein R₁, R₂, R₃, and R₄ are ethyl.

6. The method of claim 1 wherein R₁, R₂, R₃, and R₄ are methyl.

7. The method of claim 6 wherein:
R₅ is H or methyl;
R₆ is methyl substituted with benzyloxy or C₁-C₆ alkoxy; and
R₇ is methyl.

8. The method of claim 6 wherein:
R₅ is H or methyl; and
R₆ and R₇, taken together, is a cyclopropyl group.

9. The method of claim 6 wherein:
R₅, R₆ and R₇, taken together, is a furanyl group.

10. The method of claim 6 wherein:
R₅ is H; and
R₆ and R₇, taken together, is a tetrahydrofuranyl group.

11. The method of claim 6 wherein:
R₅ is H; and
R₆ and R₇, taken together, is a cyclopropyl group.

12. The method of claim 1, wherein the patient is a mammal.

13. The method of claim 1, wherein the patient is a human.

14. The method of claim 1, further comprising administering one or both of an antioxidant or a reducing agent.

15. The method of claim 1, further comprising administering an anti-neoplastic agent.

16. The method of claim 1, further comprising administering an additional anti-angiogenic agent.

17. The method of claim 16, wherein the anti-angiogenic agent is an anti-oxidant, VEGF antagonist, bFGF antagonist, NOS antagonist, or a combination thereof.

18. A method of treating a patient having a disease state that involves angiogenesis, comprising:
administering to the patient a hydroxylamine compound or a derivative thereof having formula I in a therapeutically sufficient amount to inhibit pathological angiogenesis;
wherein formula I is: 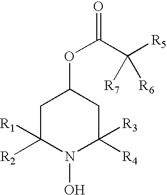
wherein R₁ and R₂ are, independently, H or C₁ to C₃ alkyl;
R₃ and R₄ are, independently, C₁ to C₃ alkyl;
R₅ is H, OH, or C₁ to C₆ alkyl;
R₆ is or C₁ to C₆ alkyl, alkenyl, alkynyl, or substituted alkyl or alkenyl;
R₇ is C₁ to C₆ alkyl, alkenyl, alkynyl, or substituted alkyl or alkenyl;
wherein R₁ and R₂, taken together, or R₃ and R₄, taken together, or both are cycloalkyl;
wherein R₆ and R₇, or R₅, R₆ and R₇, taken together, form a carbocycle or heterocycle comprising a 3 to 7 membered ring.

19. The method of claim 18, wherein the hydroxylamine compound is:

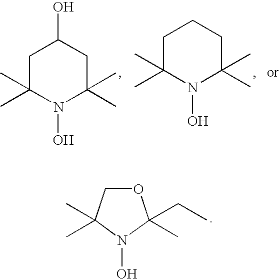

20. The method of claim 18, wherein the hydroxylamine compound is:

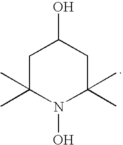

21. The method of claim 1 wherein R₁, R₂, R₃, and R₄ are a C₁-C₃ alkyl.

22. The method of claim 1 wherein R₁, R₂, R₃, and R₄ are ethyl.

23. The method of claim 1 wherein R₁, R₂, R₃, and R₄ are methyl.

24. The method of claim 23 wherein:
R₅ is H or methyl;
R₆ is methyl substituted with benzyloxy or C₁-C₆ alkoxy; and
R₇ is methyl.

25. The method of claim 23 wherein:
R₅ is H or methyl; and
R₆ and R₇, taken together, is a cyclopropyl group.

26. The method of claim 23 wherein:
R₅, R₆ and R₇, taken together, is a furanyl group.

27. The method of claim 23 wherein:
R₅ is H; and
R₆ and R₇, taken together, is a tetrahydrofuranyl group.

28. The method of claim 23 wherein:
R₅ is H; and
R₆ and R₇, taken together, is a cyclopropyl group.

29. The method of claim 18, wherein the patient is a mammal.

30. The method of claim 18, wherein the patient is a human.

31. The method of claim 18, wherein the disease state is characterized by neoplasm.

32. The method of claim 31, wherein the disease state is a cancer.

33. The method of claim 32, wherein the disease state is fibrosarcoma, myxosarcoma, liposarcoma, chondrosarcoma, osteogenic sarcoma, chordoma, angiosarcoma, endotheliosarcoma, lymphangiosarcoma, lymphangioendotheliosarcoma, synovioma, mesothelioma, Ewing's tumor, leiomyosarcoma, rhabdomyosarcoma, colon carcinoma, pancreatic cancer, breast cancer, ovarian cancer, prostate cancer, squamous cell carcinoma, basal cell carcinoma, adenocarcinoma, sweat gland carcinoma, sebaceous gland carcinoma, papillary carcinoma, papillary adenocarcinomas, cystadenocarcinoma, medullary carcinoma, bronchogenic carcinoma, renal cell carcinoma, hepatoma, bile duct carcinoma, choriocarcinoma, seminoma, embryonal carcinoma, Wilms' tumor, cervical cancer, testicular tumor, lung carcinoma, small cell lung carcinoma, bladder carcinoma, epithelial carcinoma, glioma, astrocytoma, medulloblastoma, craniopharyngioma, ependymoma, pinealoma, hemangioblastoma, acoustic neuroma, oligodendroglioma, meningioma, melanoma, neuroblastoma, retinoblastoma, acoustic neuroma, neurofibroma, trachoma, or pyogenic granulomas.

34. The method of claim 18, further comprising administering an antioxidant or a reducing agent.

35. The method of claim 31, further comprising administering an anti-neoplastic agent.

36. The method of claim 18, further comprising administering an additional anti-angiogenic agent.

37. The method of claim 36, wherein the additional anti-angiogenic agent is an anti-oxidant, VEGF antagonist, bFGF antagonist, NOS antagonist, or a combination thereof.
